(19) United States
(12) Patent Application Publication
(10) Pub. No.: US 2022/0031628 A1
(43) Pub. Date: Feb. 3, 2022

(54) NANOPARTICLES FOR PREPARING REGULATORY B CELLS

(71) Applicants: INSTITUT NATIONAL DE LA SANTE ET DE LA RECHERCHE MEDICALE (INSERM), Paris (FR); ASSISTANCE PUBLIQUE - HOPITAUX DE PARIS, Paris (FR); UNIVERSITÉ DE PARIS, Paris (FR); UNIVERSITE PARIS 13, Villetaneuse (FR); CENTRE NATIONAL DE LA RECHERCHE SCIENTIFIQUE (CNRS), Paris (FR)

(72) Inventors: Chloé DUBREIL, Paris Cedex 15 (FR); Laurence MOTTE, Bobigny (FR); Peter VAN ENDERT, Paris (FR)

(21) Appl. No.: 17/297,207

(22) PCT Filed: Nov. 27, 2019
(86) PCT No.: PCT/EP2019/082665

§ 371 Date: May 26, 2021

(30) Foreign Application Priority Data
Nov. 27, 2018 (EP) 18306575.4

(51) Int. Cl. A61K 9/51; A61K 39/00; C12N 5/0781; A61K 35/17

(52) U.S. Cl. CPC A61K 9/5146; A61K 39/0008; A61K 2039/55555; A61K 35/17; C12N 5/0635

(57) ABSTRACT

The present invention relates to nanoparticles, kits, methods and compositions which are suitable for increasing the number of B regulatory (B_{reg}) cells in a population of B cells; for producing Interleukin-10 (IL-10) or TGF-β. The inventors have shown that biocompatible nanoparticles comprising an antigen may be used for inducing B regulatory (B_{reg}) cells. This production, either ex vivo, or in vivo, or in vitro, was associated to temporary or lasting remission of disease in spontaneously diabetic NOD mice.

### FIELD OF THE INVENTION

The present invention relates to nanoparticles, methods for increasing the number of B regulatory (B_{reg}) cells in a population of B cells or for producing Interleukin-10 (IL-10), or for producing Transforming Growth Factor beta (TGF-β), to compositions and kits.

### BACKGROUND OF THE INVENTION

Auto-immune disorders are a major public health concern, as they encompass a broad category of related diseases, in which the person's immune system attacks his or her own tissue. The origin of such auto-immune disorders is not always identified. However, they may result in risks of secondary complications and a reduced life expectancy. For instance, in the case of autoimmune type-1 diabetes (T1D), it results in life-long dependence on insulin injections. Still, preventive or curative strategies with good efficacy in patients having an autoimmune disorder such as T1D, are lacking.

Given evidence for a significant remaining beta cell mass at clinical disease onset in many patients and the availability of autoantibody and genetic screening methods for identifying individuals at high risk of autoimmune disorders, there is still a window of opportunity for strategies preventing disease or inducing remission.

Although the initial events triggering the process at the origin of autoimmune disorders are poorly understood, in the context of T1D, it is clear that the disease results from a dysregulation of the islet-specific adaptive immune response in which physiological mechanisms of tolerance are overwhelmed by an autoaggressive response targeting beta cell antigens.

Dysregulation involves autoreactive T cells that expand in number and in the array of self-antigens recognized, acquire higher avidity and become resistant to the action of regulatory T cells (T_{reg}) that normally suppress harmful autoreactivity via regulatory cytokines or cell-cell contact. Although less studied and understood, B lymphocytes also can play both pathogenic and protective roles in autoimmune disorders, by presenting beta cell antigens captured through their cell surface receptor to autoreactive T cells, or by producing regulatory cytokines such as IL-10.

For instance, strategies of immunointervention in T1D aim to restore the physiological dominance of adaptive tolerance to beta cells. This can be done either by immunomodulation using agents targeting broad immune cell populations, or by antigen-specific strategies aiming to restore tolerance to specific beta cell antigens which is then hoped to spread to other antigens through the phenomenon of “infectious tolerance”.

One such approach is to combine an antigen (such as an auto-antigen) with a drug or small molecule with a tolerogenic effect.

Yeste et al. («Nanoparticle-mediated codelivery of myelin antigen and a tolerogenic small molecule suppresses experimental autoimmune encephalomyelitis»; 2012; Proc. Natl. Acad. Sci. USA 109, 11270-11275) and Yeste et al. (bis) («Tolerogenic nanoparticles inhibit T cell-mediated autoimmunity through SOCS2»; 2016; Sci. Signaling, Vol. 9,433) report a strategy where tolerogenic nanoparticles (NPs) are coupled to an antigen and administered in mouse models of multiple sclerosis, and pre-diabetic mice. The authors suggest that approach could modulate in vivo the activation of regulatory T cells (T_{regs}) by dendritic cells.

WO2016154362 further suggests to directly induce regulatory T cells from a population of T cells by functionalized nanoparticles, and then to administer the preparation for preventing or treating T1D.

Other nanoparticles have been established as multifunctional nanoplatforms, in particular for the diagnosis of T1D in Dubreil et al. (“Tolerogenic Iron Oxide Nanoparticles in Type 1 Diabetes: Biodistribution and Pharmacokinetics Studies in Nonobsese Diabetic (NOD) mice”; 2018; Small, 1802053).

Interleukin 10 (IL-10) is an anti-inflammatory, regulatory, cytokine which has been related to the induction of regulatory T cells, and which can be produced by some B cells. Alternative approaches have been reported, which instead rely on the production of regulatory B cells (B_{regs}) as a IL-10 producing B cell subset.

Kleffel et al. (“Interleukin-10+ Regulatory B Cells Arise Within Antigen-Experienced CD40+ B Cells to Maintain Tolerance to Islet Autoantigens”; 2015; Diabetes, Vol. 64) suggests to administer IL-10+ B Cells to maintain normoglycemia in NOD mice models of type-1 diabetes.

WO2018013897 suggests to treat an immune disorder by administering a therapeutically effective amount of a exogenously stimulated population of B_{regs}. This other strategy requires culturing isolated populations of B cells in the presence of soluble CD40 ligand, an anti-B cell receptor (anti-BCR) antibody and CpG oligodeoxynucleotides (ODNs) for a period of time sufficient to produce a stimulated population of B_{regs}.

Still, there remains a need for novel methods for treating or preventing immune disorders, especially auto-immune disorders, and in particular type-1 diabetes.

There also remains a need for novel methods for producing IL-10 and TGF-β, either in vitro, or ex vivo, or in vivo.

The invention has for purpose to meet the above-mentioned needs.

### SUMMARY OF THE INVENTION

According to a first embodiment, the invention relates to an in vitro or ex vivo method for increasing the number of B regulatory (B_{reg}) cells in a population of B cells, the method comprising:

(i) providing a population of isolated B cells;

(ii) bringing into contact the population of isolated B cells with an efficient amount of a biocompatible nanoparticle comprising at least one antigen, thereby increasing the number of B_{reg} cells in the population, thereby providing a B_{reg} cells-enriched composition;

(iii) optionally recovering B_{reg} cells from the B_{reg} cells-enriched composition.

According to a second embodiment, the invention relates to an in vitro or ex vivo method for producing Interleukin-10 (IL-10) or TGF-β, the method comprising:

(i) providing a population of isolated B cells;

(ii) bringing into contact the population of isolated B cells with an efficient amount of a biocompatible nanoparticle comprising at least one antigen, thereby producing Interleukin-10 or TGF-β;

(iii) optionally recovering the Interleukin-10 or TGF-β from step (ii).

According to a third embodiment, the invention relates to a composition containing a biocompatible nanoparticle comprising at least one antigen, as defined above; in combination with a population of isolated B cells.

According to a fourth embodiment, the invention relates to a kit comprising: a biocompatible nanoparticle comprising at least one antigen, as defined above; and a population of isolated B cells.

According to a fifth embodiment, the invention relates to a B_{reg} cells-enriched composition obtained by any one of the methods defined above, or recovered B_{reg} cells thereof.

According to a sixth embodiment, the invention relates to a biocompatible nanoparticle comprising at least one antigen, as defined above; for use in a method for producing B regulatory (B_{reg}) cells in vivo or for producing Interleukin-10 (IL-10) or TGF-β in vivo.

### BRIEF DESCRIPTION OF THE FIGURES

FIG. 1. NP treatment on B cells ex vivo. Splenic B cells were sorted from female NOD and C57BL/6 mice and incubated with NPs during 3 days. For each figure, in the x-axis, the data correspond from left to right to an ex vivo treatment with “None” (control), PEG-functionalized nanoparticles “PEG”, PEG-ITE, PEG-P3UmPI and PEG-ITE-P3UmPI. (A) B cells from C57BL/6 (triangle) and NOD (circle) mice were compared with respect to production of IL-10. (B) NOD Rag^{-/-} mice (n=5 per group) were injected with sorted splenic T cells obtained from newly diabetic NOD mice alone, or together with splenic B cells from prediabetic NOD mice injected 3 times with PEG-coated NPs or with ITE-P3UmPI-loaded NPs, or together with B cells treated ex vivo with the latter NPs. n=3 to 6 from 3 independent experiments. Group mean+/−SD values were compared using the two-way ANOVA test. Diabetes incidence with n=5 per group was compared with the log-rank test. Curves from the left to the right: (i) T only, (ii) T+B (PEG in vivo), (iii) T+B (PEG-ITE-P3UmPI in vivo), (iv) T+B (PEG-ITE-P3UmPI ex vivo).

FIG. 2. NP treatment on B cells ex vivo. (A) shows the effect of treatment on surface level of CD86 on NOD B cells. In (B) through (D), B cells from C57BL/6 (triangle) and NOD (circle) mice were compared with respect to production of LAP (TGF-β) (B), IL-4 (C) and IDO (D), determined by intracellular staining. The data in panels (E) indicate the effect of treatment on the percentage of follicular NOD B cells.

FIG. 3. Differential Effect on FoB and MZB. The data in panels (A) through (D) indicate the expression of cytokines (respectively IL-10, LAP, IL-4 and IDO as determined in the y-axis) by follicular (FoB) and marginal (MZB) zone NOD B cells.

### DETAILED DESCRIPTION OF THE INVENTION

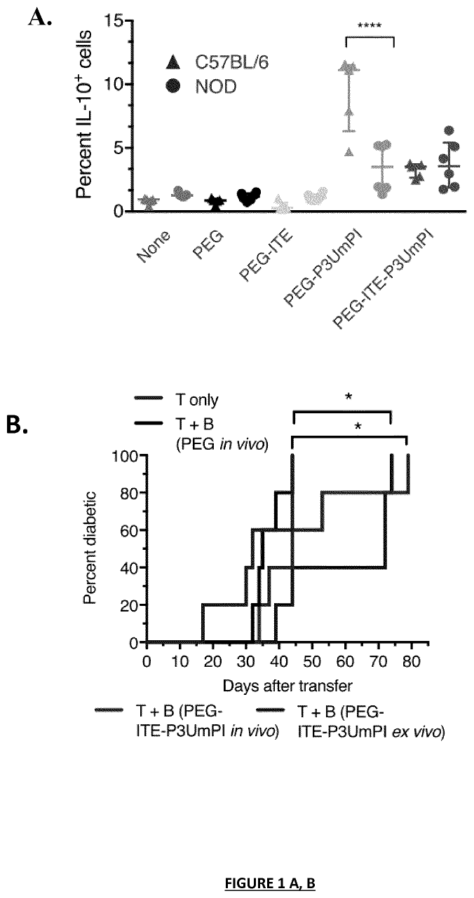

Surprisingly, the inventors have now shown that biocompatible nanoparticle comprising an antigen, were able to induce the production of regulatory B cells ex vivo. In particular, the inventors have shown, as set forth in the Examples and on FIG. 1 that ex vivo incubation of sorted splenic B cells with the particles results in rapid production of B_{regs} that transfer protection from disease in adoptive transfer experiments. It is also surprising that a strong effect on B_{regs} was observed after in vivo treatment.

Those results contrast with previous reports, which did not specifically report that the therapeutic effect of nanoparticles may also include early induction of splenic regulatory B cells (B_{regs}), in the context of a treatment of type-1 diabetes. Indeed, having considered previous reports on the effect of tolerogenic nanoparticles in murine models of multiple sclerosis and T1D, it was expected that the reported increase in the proportion of Foxp3⁺ T_{regs} would result mechanically from a reduction in the number of non-T cells rather than from their expansion.

Thus, it is proposed herein that nanoparticles comprising an antigen, such as an autoantigen, can now be used to produce antigen-specific B_{regs} ex vivo, a feature of interest in immune diseases, especially autoimmune diseases, and other contexts where induction of active B cell tolerance is desirable.

In particular, the strongest effect of biocompatible nanoparticles functionalized with a diabetes autoantigen clearly targeted B lymphocytes. This effect was evident both upon a 10-day treatment of pre-diabetic mice and upon a 3-day in vitro treatment of sorted B cells and included B cell activation, expansion of follicular B cells and production of regulatory B cells producing IL-10, TGF-β, IL-4 and Indoleamine deoxygenase (IDO).

Also, it is found herein that B cell expansion and conversion to regulatory B cells concerns mainly the follicular B cell subset. However, a large variety of B cells at different stages of differentiation and maturation have been identified as regulatory B cells, so that it is reasonable to speculate that almost any of the B cells, in the sense of the invention, may be able to produce IL-10, the hallmark cytokine of B_{regs}, but also TGF-β, under appropriate conditions.

Without wishing to be bound by the theory, it is also likely that B cell interactions with other cells or soluble mediators in vivo may direct IL-10 or TGF-β expression, as B cells expressing TGF-β on day-3 may convert to IL-10 production on day-10.

According to a first embodiment, the invention relates to an in vitro or ex vivo method for increasing the number of B regulatory (B_{reg}) cells in a population of B cells, the method comprising:

(i) providing a population of isolated B cells;

(ii) bringing into contact the population of isolated B cells with an efficient amount of a biocompatible nanoparticle comprising at least one antigen, thereby increasing the number of B_{reg} cells in the population, thereby providing a B_{reg} cells-enriched composition;

(iii) optionally recovering B_{reg} cells from the B_{reg} cells-enriched composition.

According to a second embodiment, the invention relates to an in vitro or ex vivo method for producing Interleukin-10 (IL-10) or TGF-β, the method comprising:

(i) providing a population of isolated B cells;

(ii) bringing into contact the population of isolated B cells with an efficient amount of a biocompatible nanoparticle comprising at least one antigen, thereby producing Interleukin-10 or TGF-β;

(iii) optionally recovering the Interleukin-10 or TGF-β from step (ii).

In a non-exhaustive manner, such populations of isolated B cells may be follicular (FoB) and marginal (MZB) zone B cells.

Such populations of isolated B cells may have been previously obtained either from an individual not having, or not presumed to have, an immune disorder (i.e. an autoimmune disorder).

Alternatively, such populations of isolated B cells may have been previously obtained either from an individual having, or presumed to have, an immune disorder (i.e. an autoimmune disorder).

According to a particular embodiment of said methods, the at least one antigen is an autoantigen.

According to a particular embodiment, the at least one antigen is a diabetes autoantigen selected from: insulin, preproinsulin, proinsulin, or an immunologically active fragment thereof.

According to a particular embodiment, the biocompatible nanoparticle is a tolerogenic biocompatible nanoparticle.

According to a particular embodiment, the biocompatible nanoparticle is a tolerogenic biocompatible nanoparticle further comprising at least a ligand which can bind to an aryl hydrocarbon receptor (AHR) transcription factor.

According to the said particular embodiment, the ligand which can bind to an aryl hydrocarbon receptor (AHR) transcription factor is 2-(1′H-indole-3′-carbonyl)-thiazole-4-carboxylic acid methyl ester (ITE).

According to a particular embodiment the said nanoparticle has an average size of less than about 60 nm, such as less than about 50 nm; and preferably less than 20 nm.

According to a third embodiment, the invention relates to a composition containing a biocompatible nanoparticle comprising at least one antigen, as defined above; in combination with a population of isolated B cells.

According to a fourth embodiment, the invention relates to a kit comprising:
a biocompatible nanoparticle comprising at least one antigen, as defined above; and
a population of isolated B cells.

According to a fifth embodiment, the invention relates to a B_{reg} cells-enriched composition obtained by any one of the methods defined above, or recovered B_{reg} cells thereof. Therapeutically effective amounts of regulatory B cells can be administered by a number of routes, including parenteral administration, for example, intravenous, intraperitoneal, intramuscular, intrasternal, or intraarticular injection, or infusion. Preferably, the biocompatible nanoparticle comprising at least one antigen is present in an injectable solution.

The regulatory B cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of regulatory B cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ regulatory B cells/m². In a certain embodiment, the dose used in the treatment of human subjects ranges from about 3.8×10⁹ to about 3.8×10¹⁰ regulatory B cells/m². In additional embodiments, a therapeutically effective amount of regulatory B cells can vary from about 5×10⁶ cells per kg body weight to about 7.5×10⁸ cells per kg body weight, such as about 2×10⁷ cells to about 5×108 cells per kg body weight, or about 5×10⁷ cells to about 2×10⁸ cells per kg body weight. The exact amount of regulatory B cells is readily determined by one of skill in the art based on the age, weight, sex, and physiological condition of the subject. Effective doses can be extrapolated from dose-response curves derived from in vitro or animal model test systems

A regulatory B cell-enriched population, or composition thereof, can include at least 5%, at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 99%, or 100% regulatory B cells that produce IL-10. For instance, a regulatory B cell-enriched population, or composition thereof may have at least a 2-fold, or 5-fold, or 10-fold, or 100-fold, 1000-fold increase of B_{reg} cells over a reference non-enriched composition.

According to a particular embodiment, the B_{reg} cells-enriched composition, or recovered B_{reg} cells thereof are for use as a medicament. Hence, the B_{reg} cells-enriched composition, or recovered B_{reg} cells thereof can be used for the preparation of a pharmaceutical composition.

According to a sixth embodiment, the invention relates to a biocompatible nanoparticle comprising at least one antigen, as defined above; for use in a method for producing B regulatory (B_{reg}) cells in vivo or for producing Interleukin-10 (IL-10) or TGF-β in vivo.

Hence, the invention also relates to a method for producing B regulatory (B_{reg}) cells in vivo or for producing Interleukin-10 (IL-10) in vivo, or for producing TGF-β in vivo, which comprises a step of administering a B_{reg} cells-enriched composition obtained by any one of the methods defined above, or recovered B_{reg} cells thereof to an individual in need thereof.

The invention also relates to a method for producing B regulatory (B_{reg}) cells in vivo or for producing Interleukin-10 (IL-10) in vivo, or for producing TGF-β in vivo, which comprises a step of administering a biocompatible nanoparticle comprising at least one antigen, or a composition thereof, to an individual in need thereof

Advantageously, a B_{reg} cells-enriched composition as defined herein may be administered, or co-administered, with a population of T cells; i.e. regulatory T cells (T_{regs}) or a T_{reg} cells-enriched composition.

### Definitions

As used herein, the terms “B regulatory cells” or “Bregs” refer to a particular sub-set of B lymphocytes. Such B regulatory cells are generally defined as IL-10 producing (IL-10⁺) B cells. Such B regulatory cells may also be expressing TGF-β (TGF-β+). They may be further characterized as previously defined in Kleffel et al. («Interleukin-10+ Regulatory B Cells Arise Within Antigen-Experienced CD40+ B Cells to Maintain Tolerance to Islet Autoantigens»; Diabetes; 2015). Hence, the term «B regulatory cells» may correspond to IL-10⁺ and/or TGF-β⁺ B cells (preferably IL-10⁺) having at least one of the following phenotypes:
CD19+/CD27+/CD24+;
CD19+/CD27−/CD24+/CD38+;
CD19+/CD1d+/CD5+
CD19+/CD21+/IgM+/CD23+
CD19+/Tim-1+.

As used herein, the terms “effective amount” and “effective to treat (or prevent)” as used herein, refer to an amount or a concentration of one or more of the compositions described herein utilized for a period of time (including acute or chronic administration and periodic or continuous administration) that is effective within the context of its administration for causing an intended effect or physiological outcome.

As used herein, the term “subject” or “patient” may encompass an animal, human or non-human, rodent or non-rodent. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

As used herein, a “diagnosis” may also encompass the “follow-up” of a given patient or population of patients over time. When the patient was not previously diagnosed, this term may also encompass the “detection” of type-1 diabetes.

As used herein, the singular form “a”, “an” and “the” include plural references unless the context clearly dictates otherwise. For example, the term “a pharmaceutically acceptable carrier” encompasses a plurality of pharmaceutically acceptable carriers, including mixtures thereof.

As used herein, «a plurality of» may thus include «two» or «two or more».

As used herein, «comprising» may include «consisting of».

As used herein, an “immunologically active fragment” generally refers to a fragment of a given antigen (e.g. preproinsulin or proinsulin) having at least five (5) consecutive amino acids from the said antigen. Thus, this definition may encompass fragments having at least 5, 6, 7, 8, 9, 10, 11, 12, 13, 14, 15, 16, 17, 18, 19, 20, 21, 22, 23, 24, 25, 26, 26, 27, 28, 29, or 30 consecutive aminoacids from the said antigen.

As used herein, the term “biocompatible” is meant to refer to compounds (e.g. nanoparticles) which do not cause a significant adverse reaction in a living animal when used in pharmaceutically relevant amounts.

As used herein, a “pharmaceutically acceptable carrier” is intended to include any and all carrier (such as any solvents, dispersion media, coatings, antibacterial and antifungal agents, isotonic and absorption delaying agents, and the like) which is compatible with pharmaceutical administration. The use of such media and agents for pharmaceutically active substances are known. Except insofar as any conventional media or agent is incompatible with the active compound, such media can be used in the compositions of the invention.

As used herein, the term “nanoparticles” is meant to refer to particles having an average size (such as a diameter, for spherical or nearly spherical nanoparticles) of 100 nanometres (nm) in size or less. The “diameter” is typically defined as the “crystalline diameter” or as the “hydrodynamic diameter”. The crystalline size (or “diameter” if applicable) of a population of nanoparticles can be determined herein by transmission electron microscopy whereas the hydrodynamic size related to surface functionalization is measured by dynamic laser light scattering (DLS), in a physiological medium, for example NaCl 0.9%, NaCl 0.9%/Glucose 5%, or other buffer media at a physiological pH, used for biological evaluation as well as in vitro and in vivo experiments, as described in the Material & Methods section.

As a general reference, the average hydrodynamic size (or “diameter”) is most preferably determined in a physiological medium corresponding to NaCl 0.9%/Glucose 5% at pH 7.4 and 37° C. Even though spherical nanoparticles are particularly considered in the context of the invention, it will be understood herein that the term “nanoparticle” is not meant to refer exclusively to one type of shape. Accordingly, this term may also encompass other shapes, selected from: spherical nanoparticles, rod-shaped nanoparticles, vesicle-shaped nanoparticles, and S-shaped worm-like particles as described in Hinde et al. (“Pair correlation microscopy reveals the role of nanoparticle shape in intracellular transport and site of drug release”; Nature nanotechnology; 2016) as well as other morphologies such as nanoflower, raspberry, and core-shell nanoparticles.

Nanoparticles according to the invention may include one or more ligands (i.e. targeting moieties), in addition to the considered antigen, such as one or more ligand(s) which can bind to an aryl hydrocarbon receptor (AHR) transcription factor.

As used herein, “Aryl hydrocarbon receptor (AhR)” refers to a transcription factor that upon activation by its ligand 2-(1 [symbol] H-indole-3 [symbol] -carbonyl)-thiazole-4-carboxylic acid methyl ester (ITE) or other ligands induces tolerogenic dendritic cells (DCs) that promote the generation of regulatory T cells. AhR is a basic helix-loop-helix/PAS domain containing ligand-activated transcription factor that, once activated, can bind to specific DNA motif sequences (called xenobiotic response elements or XREs) and initiate transcription, as described in Nebert et al (J Biol Chem 279(23):23847-23850, 2004).

Hence, as used herein, a “ligand which can bind to an aryl hydrocarbon receptor (AHR) transcription factor» refers to a ligand (for instance, a naturally-occurring, recombinant or synthetic polypeptide) which can bind to the Aryl hydrocarbon Receptor, in a manner susceptible to activate the Aryl hydrocarbon Receptor and generate a tolerogenic signal in Antigen-Presenting Cells (APC), such as dendritic cells (DC). Particular examples of such ligands are described hereafter.

As used herein, the terms “targeting moiety” and “targeting agent” are used interchangeably and are intended to mean any agent, such as a functional group, that serves to target or direct the nanoparticle to a particular location or association (e.g., a specific binding event). Targeting moieties generally target cell surface receptors. For example, a targeting moiety may be used to target a molecule to a specific target protein, or to a particular cellular location, or to a particular cell type, to selectively enhance accumulation of the nanoparticle. Suitable targeting moieties include, but are not limited to, polypeptides, nucleic acids, carbohydrates, lipids, hormones including proteinaceous and steroid hormones, growth factors, receptor ligands, antigens and antibodies, and the like. For example, as is more fully outlined below, the nanoparticles of the invention may include a targeting moiety to target the nanoparticles (including biologically active agents associated with the nanoparticles) to a specific cell type, such as liver, spleen, pancreas or kidney cell type.

As used herein, the term “lipid” includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

As used herein, the term “linked to”, such as in “a ligand linked to the nanoparticles” or “a ligand conjugated to the nanoparticles” may refer either to a covalent link or to a non-covalent link. In a non-limitative manner, such non-covalent interactions may occur due to electrostatic interactions, Van der Walls forces, π-effects, and hydrophobic effects. Alternatively, covalent-interactions occur as a consequence of the formation of a covalent bond, such as the coupling of a ligand which can bind to an aryl hydrocarbon receptor (AHR) transcription factor, and a functional (reactive) chemical group at the surface of the nanoparticle. Most preferably, such ligands (i.e. targeting moieties) are present at the surface of the considered nanoparticle.

As used herein, the term “tolerogenic” is meant to refer to compounds (e.g. nanoparticles) which are able to induce immune tolerance where there is pathological or undesirable activation of the normal immune response.

As used herein, the terms “magnetic” and “superparamagnetic” is meant to refer to magnetic and superparamagnetic behavior at room temperature.

As used herein, “treating” means any manner in which one or more of the symptoms of a disease or disorder are ameliorated or otherwise beneficially altered. As used herein, amelioration of the symptoms of a particular disorder refers to any lessening of the symptoms, whether permanent or temporary, lasting or transient, that can be attributed to or associated with treatment by the compositions and methods of the present invention. Accordingly, the expression “treating” may include “reversing partially or totally the effect” of a given condition, or even “curing” when permanent reversal is considered. It flows from the above that, in the context of a “treatment of type-1 diabetes”, this term shall be interpreted to encompass the treatment of a subject/patient, or of a group of subjects/patients, which actually have, or are presumed to have, type-1 diabetes. However it does necessarily flow that the targeted patients are all at the same stage of the disease. Accordingly, the present invention is not restricted to the treatment of patients or groups of patients which are at a late stage of the disease, but it may also concern patients or groups of patients at an early stage of the disease.

As used herein, “preventing” encompasses “reducing the likelihood of occurrence” and “reducing the likelihood of re-occurrence”.

As used herein an “autoimmune disorder” or “autoimmune disease” may refer to any condition which arises from and/or is directed against an individual's own tissues, or a co-gregate or manifestion thereof or resulting condition therefrom. Examples of autoimmune diseases or disorders include, but are not limited to: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease.

As used herein, the term “type-1 diabetes”, or “insulin-dependent diabetes”, refers to any form of diabetes which can be characterized by a deficient, or insufficient, insulin production, as defined by the World Health Organization (see Diabetes Fact sheet N^{o} 312). For instance, this term may encompass patients resulting from the pancreas's failure to produce enough insulin, whether the cause is known or unknown. This term may also encompass patients still having a normal fasting glycaemia but developing a type-1 diabetes, for instance because they harbour functionally impaired and/or a reduced mass of insulin-producing beta cells in the pancreatic islets. This term may also encompass patients having an impaired fasting glycaemia thus having a clinically manifest type-1 diabetes, as discussed above. On the other hand, the population of patients characterized by the occurrence of “type-1 diabetes” does not encompass “type-2 diabetes” or “gestational diabetes”, or intermediate conditions referred as “impaired glucose tolerance (IGT)” or “impaired fasting glycaemia (IFG)” which are not associated with type-1 diabetes.

As used herein, “treating a type-1 diabetes” may thus comprise “reducing, arresting, reversing partially or totally the loss of insulin producing beta cells of the pancreatic islets, whether directly or indirectly”. It may also include the symptomatic treatment of type-1 diabetes, including “normalizing and/or reducing glycemia” in a type-1 disease patient.

For groups of subjects who do not have, or are not presumed to have, type-1 diabetes, the term “preventing” is preferred.

Nanoparticles of the Invention

Nanoparticles (NPs) of the invention are biocompatible nanoparticle comprising at least one antigen, such as an auto-antigen, preferably a diabetes autoantigen.

According to one particular embodiment, nanoparticles (NPs) of the invention are biocompatible nanoparticles further comprising (or be linked to) an IgG binding moiety, such as an IgG binding moiety comprising (or even consisting of) at least two (such as two or three) IgG binding domains.

Such an IgG binding moiety may consist of an IgG binding moiety from streptococcal protein G, such as an IgG binding moiety comprising (or even consisting of) at least two (such as two or three) IgG binding domains of streptococcal protein G placed in tandem arrangement. The nanoparticle may be covalently or non-covalently bound to said IgG binding moiety

According to one (non-mutually exclusive) particular embodiment, nanoparticles (NPs) of the invention are biocompatible tolerogenic nanoparticles.

According to one particular embodiment, nanoparticles (NPs) of the invention are biocompatible tolerogenic nanoparticle comprising at least: (i) a ligand which can bind to an aryl hydrocarbon receptor (AHR) transcription factor; and (ii) an antigen, such as an auto-antigen, and preferably a diabetes autoantigen.

Most preferably, the ligand which can bind to an aryl hydrocarbon receptor (AHR) transcription factor is the tolerogenic AhR ligand 2-(1′H-indole-3′-carbonyl)-thiazole-4-carboxylic acid methyl ester (ITE).

Most preferably, the diabetes autoantigen is a polypeptide comprising a sequence selected from the group consisting of preproinsulin or an immunologically active fragment thereof; such as an immunologically active fragment of proinsulin.

This ligand and antigen (i.e. diabetes autoantigen) may be either comprised (e.g. encapsulated) within the nanoparticle or attached (linked covalently or non-covalently) in a matter suitable for release into and/or contact with the surrounding medium (i.e. at the surface of the nanoparticle).

Most preferably, the nanoparticles are attached (linked covalently or non-covalently) to the AhR ligands and the antigen (i.e. diabetes autoantigen) described herein (e.g. via functional groups). When applicable, such functional groups may be born by a polymer such as, but not limited to, polyethylene glycol (PEG).

Thus, a nanoparticle of the invention may be linked covalently or non-covalently to at least one antigen, such as at least one auto-antigen.

Thus, a nanoparticle of the invention may be linked covalently or non-covalently to at least: (i) a ligand which can bind to an aryl hydrocarbon receptor (AHR) transcription factor; and (ii) a diabetes autoantigen.

According to another exemplified embodiment, the nanoparticle may be:
covalently bound to a ligand which can bind to an aryl hydrocarbon receptor (AHR) transcription factor; and
non-covalently bound to a diabetes autoantigen.

According to some other embodiments, the nanoparticle may be:
covalently bound to a ligand which can bind to an aryl hydrocarbon receptor (AHR) transcription factor; and
covalently bound to an antigen (i.e. a diabetes autoantigen).

According to some embodiments, the nanoparticle may be:
covalently bound to a ligand which can bind to an aryl hydrocarbon receptor (AHR) transcription factor; and
non-covalently bound to an antigen (i.e. a diabetes autoantigen).

According to some other embodiments, the nanoparticle may be:
non-covalently bound to a ligand which can bind to an aryl hydrocarbon receptor (AHR) transcription factor; and
covalently bound to an antigen (i.e. a diabetes autoantigen).

According to some other embodiments, the nanoparticle may be:
non-covalently bound to a ligand which can bind to an aryl hydrocarbon receptor (AHR) transcription factor; and
non-covalently bound to an antigen (i.e. a diabetes autoantigen).

Also, the nanoparticle may be covalently bound to an IgG binding moiety; and non-covalently bound to an antigen (i.e. an autoantigen such as a diabetes autoantigen).

Hence, the nanoparticle may be covalently bound to an IgG binding moiety; and covalently bound to an antigen (i.e. an autoantigen such as a diabetes autoantigen).

Alternatively, the nanoparticle may be non-covalently bound to an IgG binding moiety; and covalently bound to an antigen (i.e. an autoantigen such as a diabetes autoantigen).

Alternatively, the nanoparticle may be non-covalently bound to an IgG binding moiety; and non-covalently bound to an antigen (i.e. an autoantigen such as a diabetes autoantigen).

According to some embodiments, nanoparticles of the invention are magnetic nanoparticles (i.e. nanoparticles having superparamagnetic properties). For instance, nanoparticles of the invention may be metal-oxide nanoparticles (i.e. ultrasmall superparamagnetic iron-oxide (USPIO) nanoparticles).

The nanoparticles which are particularly considered, and useful in the methods and compositions described herein, are made of materials that are (i) biocompatible e.g. do not cause a significant adverse reaction in a living animal when used in pharmaceutically relevant amounts; (ii) feature functional groups to which the binding moiety can be covalently attached, (iii) exhibit low non-specific binding of interactive moieties to the nanoparticle, and (iv) are stable in solution, e.g., the nanoparticles do not precipitate.

The nanoparticles can be monodisperse (a single crystal of a material, e.g., a metal, per nanoparticle) or polydisperse (a plurality of crystals, e.g., 2, 3, or 4, per nanoparticle).

A number of biocompatible nanoparticles are known in the art, e.g., organic or inorganic nanoparticles. Liposomes, dendrimers, carbon nanomaterials and polymeric micelles are examples of organic nanoparticles. Quantum dots can also be used. Inorganic nanoparticles include metallic nanoparticle, e.g., Au, Ni, Pt and TiO₂ nanoparticles. Magnetic nanoparticles can also be used, e.g., spherical nanocrystals of 10-20 nm with a Fe2+ and/or Fe3+ core surrounded by dextran or PEG molecules.

Metal-oxide nanoparticles, such as iron-oxide nanoparticles, are also considered. In some embodiments, colloidal gold nanoparticles can be used, e.g., as described in Qian et al. (Nat. Biotechnol. 26(1):83-90 (2008)); U.S. Pat. Nos. 7,060,121; 7,232,474; and US2008/0166706. Suitable nanoparticles, and methods for constructing and using multifunctional nanoparticles, are discussed in e.g., Sanvicens and Marco (Trends Biotech., 26(8): 425-433 (2008)).

Spherical nanoparticles are particularly considered. Alternatively, non-spherical nanoparticles are also considered, as described hereafter.

According to some other embodiments, the nanoparticles of the invention may also include micelle-shaped nanoparticles, vesicle-shaped nanoparticles, rod-shaped nanoparticles, and worm-shaped nanoparticles as described for instance in Hinde et al. (“Pair correlation microscopy reveals the role of nanoparticle shape in intracellular transport and site of drug release”; Nature nanotechnology; 2016).

A nanoparticle (or population thereof) having an average size (or «diameter») of less than about 100 nm includes nanoparticles (or populations thereof) having an average size (or «diameter») of less than about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, and 3 nm.

In some embodiments, the nanoparticles have an average size (or “diameter”) of about 1-100 nm, e.g., about 20-75 nm, e.g. about 25-75 nm, e.g., about 40-60 nm, or about 50-60 nm. The polymer component in some embodiments can be in the form of a coating, e.g., about 5 to 20 nm thick or more.

According to a most preferred embodiment, the nanoparticles have an average size (or «diameter») of less than about 60 nm, especially less than about 50 nm, especially less than about 20 nm.

In an illustrative manner, nanoparticles having an average size of about 3 nm are reported in Richard et al. (Nanomedicine (Lond) 2016. DOI 10.2217/nnm-2016-0177).

Methods for the functionalization of nanoparticles are known in the Art. Accordingly, reference is made to Perrier et al. («Methods for the Functionalisation of Nanoparticles: New Insights and Perspectives»; 2010; Chem. Eur. J. 2010, 16, 11516-11529). In a non-limitative manner, such functional groups may comprise or consist of one or more functional groups selected from: alkyl, alkenyl, alkynyl, phenyl, halo, fluoro, chloro, bromo, iodo, hydroxyl, carbonyl, aldehyde, haloformyl, carbonate ester, carboxylate, ester, methoxy, hydroperoxy, peroxy, ether, hemiacetal, hemiketal, acetal, ketal, orthoester, methylenedioxy, orthocarbonate ester, carboxalide, amine, imine, imide, azide, azo(diimide), cyanate, isocyanate, nitrate, nitrile, isonitrile, nitrosooxy, nitro, nitroso, oxime, pyridyl, sulfhydryl, sulfide, disulfide, sulfinyl, sulfonyl, sulfino, sulfothiocyanate, isothiocyanate, thiol, carbonothioyl, phosphino, phosphono, phosphate, borono, boronate, borino, and borinate functional groups.

In some embodiments, nanoparticles of the invention can be associated with a polymer that includes functional groups.

When applicable, this also serves to keep the metal oxides dispersed from each other. The polymer can be a synthetic polymer, such as, but not limited to, polyethylene glycol (PEG) or silane, natural polymers, or derivatives of either synthetic or natural polymers or a combination of these.

Useful polymers are hydrophilic. In some embodiments, the polymer “coating” is not a continuous film (i.e. a continuous film around a magnetic metal oxide), but is a “mesh” or “cloud” of extended polymer chains attached to and surrounding the metal oxide. The polymer can comprise polysaccharides and derivatives, including dextran, pullanan, carboxydextran, carboxmethyl dextran, and/or reduced carboxymethyl dextran. When applicable, the metal oxide can be a collection of one or more crystals that contact each other, or that are individually entrapped or surrounded by the polymer.

Thus, nanoparticles of the invention may also consist of porous nanoparticles such as metal organic framework (MOF) nanoparticles, which can be functionalized and used as effective carriers for drug delivery. Metal-organic frameworks, also referred herein as “porous coordination polymers (PCPs)” can be generally defined as coordination polymers of hybrid inorganic-organic framework containing metal ions and organic ligands coordinated to the metal ions. These materials are organized into one-, two- or three-dimensional frameworks where the metal clusters are bound together by spacer ligands in a periodic manner. These materials have a crystalline structure, are most often porous and are used in many industrial applications such as the storage of gas, the adsorption of liquids, the separation of liquids or gases, catalysis, and more recently medical applications.

According to one embodiment, the biocompatible nanoparticle (i.e. biocompatible tolerogenic nanoparticle) is functionalized (linked/conjugated) with phosphonate polyethylene glycol (PEG) molecules.

According to said embodiment, the biocompatible nanoparticle has, even more preferably, an average density of PEG molecules at the surface of the nanoparticle ranging from 0.1 to 5 PEG per nm², such as from 0.5 to 2 PEG per nm².

According to one embodiment, the biocompatible tolerogenic nanoparticle of the invention comprises a ligand which can bind to an aryl hydrocarbon receptor (AHR) transcription factor is 2-(1′H-indole-3′-carbonyl)-thiazole-4-carboxylic acid methyl ester (ITE). Other types of AHR ligands are further described hereafter.

According to one embodiment, the biocompatible nanoparticle is linked to the ligand which can bind to an AHR transcription factor with an average density of ligand at the surface of the nanoparticle from 0.5 to 4 ligands per nm²; the said ligand being preferably ITE.

According to one embodiment, the biocompatible tolerogenic nanoparticle is functionalized with phosphonate polyethylene glycol (PEG) molecules; and it further comprises at least one ligand which can bind to an AHR transcription factor.

According to one embodiment, the biocompatible tolerogenic nanoparticle is linked to phosphonate polyethylene glycol (PEG) molecules, and further linked to at least one ligand which can bind to an AHR transcription factor, the said ligand being preferably ITE.

According to one preferred and exemplified embodiment, the biocompatible tolerogenic nanoparticle is linked to an IgG binding moiety, such as an IgG binding moiety from streptococcal protein G, such as an IgG binding moiety consisting of at least two IgG binding domains of streptococcal protein G placed in tandem arrangement.

Antigen

Biocompatible nanoparticles, in the context of the methods of the invention, are biocompatible nanoparticles comprising an antigen. In this context, the “antigen” is a molecule which bears a motif (i.e. a linear sequence) which is susceptible to induce an immunological response. Typically, antigens are substances specifically bound by antibodies or T lymphocyte antigen receptors. They are substances that stimulate production of or are recognized by antibodies. For instance, this immunological response may result from the interaction with cell surface immunoglobulins or signals provided by splenic non-B cells. In a non-limitative manner, such antigens may be present as a polypeptide or a complex of polypeptides, and sometimes as a nucleic acid (i.e. a deoxyribonucleic or ribonucleic acid).

As used herein, the term “antigen” may also encompass “auto-antigen”, as a preferred embodiment. Hence, the term “auto-antigen” refers to a sub-type of antigens which is (“endogenously”) present in an individual, and for which the said individual may develop a decreased or suppressed tolerance. Such auto-antigens may also be born by antigen molecules which are exogenously administered. Extended lists of auto-antigens have already been associated to auto-immune disorders, and auto-immune disorder related symptoms. For instance human autoantigen databases are readily available, as described in Wang et al. (“AAgAtlas 1.0: a human autoantigen database”; Nucl. Acids Res. 2017. 45: D769-D776) and in http://biokb.ncpsb.org/aagatlas/. The database contains more than 1000 well-annotated autoantigens, determined by text-mining and manual curation, listed both by gene name and the corresponding auto-immune disease, which are incorporated herein by reference.

In a non-exhaustive manner, such auto-antigens include: Carboxypeptidase H, Chromogranin A, Glutamate decarboxylase, Imogen-38, Insulin, Insulinoma antigen-2 (IA-2) and 2β, Islet-specific glucose-6-phosphatase catalytic subunit related protein (IGRP), Proinsulin, Preproinsulin, Glutamate Decarboxylase (GAD), Zinc-Transporter 8 (ZnT8), Chromogranin A, α-enolase, Aquaporin-4, β-arrestin, Myelin basic protein (MBP), Myelin Oligodendrocytic Glycoprotein (MOG), Myelin Proteolipid Protein (PLP), Myelin Associated Glycoprotein (MAG), Myeline-associated Oligodendrocyte Basic Protein (MOBP), 2′,3′-Cyclic-nucleotide 3′-phosphodiesterase (CNPase), 5100-1310 (S100-β), nAChR, MuSK, LRP4, Citrullinated antigen, Carbamylated antigen, Collagen such as Collagen type I, Collagen type II, Collagen type III, Collagen type IV, Heat shock proteins such as 6(-kDa heat-shock protein, Human cartilage glycoprotein 39, Double-stranded DNA, La antigen, Nucleosomal histones and ribonucleoproteins (snRNP), Phospholipid-β-2 glycoprotein I complex, Poly(ADP-ribose) polymerase, Sm antigens of U-1 small ribonucleoprotein complex11, Transaldolase, Fc-part of immunoglobulins, Aggrecan G1, Aquaporin 4 (AQP-4), NMDA-receptor, AMPA-receptor, GABA receptor, Gly-receptor, Dipeptidyl aminopeptidase-like Protein 6 (DPPX), GluR5, VGKC-complex, HU, Jo, Ri, Ma1, Ma2, Zic4, CRMP5, Amphiphysin; or a immunologically active fragment thereof.

According to one embodiment, such antigen (i.e. auto-antigen) is or comprises a polypeptide, or a immunologically active fragment thereof, which contains preferably at least five amino acids from the reference polypeptide. For instance, such antigen may comprise at least five consecutive amino acids from the reference polypeptide (i.e. insulin, preproinsulin, or proinsulin).

According to some embodiments, the antigen may be in the form of a fusion protein. A “fusion protein” refers to a protein artificially created from at least two amino-acid sequences of different origins, which are fused either directly (generally by a peptide bond) or via a peptide linker. In particular, the antigen may be in the form of a fusion protein characterised in that it comprises:
an IgG binding moiety;
as a cargo moiety, a polypeptide comprising an antigen-derived sequence.

More particularly, the antigen may be in the form of a fusion protein characterised in that it comprises:
an IgG binding moiety from streptococcal protein G;
as a cargo moiety, a polypeptide comprising an antigen-derived sequence.

According to preferred and exemplified embodiments, the antigen may be in the form of a fusion protein characterised in that it comprises:
an IgG binding moiety consisting of at least two IgG binding domains of streptococcal protein G placed in tandem arrangement;
as a cargo moiety, a polypeptide comprising an antigen-derived sequence.

Especially, the cargo moiety may comprise an ubiquitin domain fused to the N-terminal or C-terminal end of the polypeptide. However, in the case wherein enhanced degradation of the polypeptide of interest (i.e. insulin, preproinsulin, proinsulin or an immunologically fragment thereof) by ubiquitin fusion is desired, the ubiquitin domain should be fused directly to the N-terminal end of the polypeptide of interest.

Peptide linkers may be employed to separate two or more of the different components of a fusion protein of the invention. In particular, peptide linkers will advantageously be inserted between the IgG binding domains in the IgG binding moiety, and between the IgG binding moiety and the cargo moiety. Peptide linkers are classically used in fusion proteins in order to ensure their correct folding into secondary and tertiary structures. They are generally from 2 to about 50 amino acids in length, and can have any sequence, provided that it does not form a secondary structure that would interfere with domain folding of the fusion protein.

According to one exemplary embodiment, the antigen is a fusion protein which is coupled to tandem immunoglobulin-binding domains from streptococcal protein G and ubiquitin. One example of such fusion protein may be as described in WO2008035217.

When the antigen is a diabetes autoantigen, it may be a polypeptide, or immunological fragment thereof, encoded by a gene selected from: ALB, ANXA2, ANXA4, CFB, CPE, CTLA4, CYP21A2, DCN, DDC, DLAT, EXOSC3, EXOSC6, FKBP1A, GAD1, GAD2, GCG, GLUL, HIRA, HSD3B1, HSPD1, ICA1, IFNG, IL1B, IL4, INS, INSR, KCNJ4, LCN1, MBP, MPP1, MYOM2, PTPRN, PTPRN2, REG1A, SLC2A2, SLC30A8, SPP1, SST, TG, TGM2, TNF, TP73, TPO, TRIM21, TROVE2, TSHR.

When the antigen is a diabetes autoantigen, it is preferably a polypeptide comprising a sequence selected from the group consisting of preproinsulin, proinsulin, or an immunologically active fragment thereof.

As known in the Art, preproinsulin corresponds to proinsulin with a signal peptide attached to its N-terminus.

When the antigen is a diabetes autoantigen, according to one preferred embodiment, the diabetes autoantigen is, or comprises, a polypeptide comprising a sequence selected from the group consisting of insulin, preproinsulin, proinsulin, or an immunologically active fragment thereof.

Thus, the antigen may be a polypeptide comprising at least five consecutive amino acids from insulin, preproinsulin, or proinsulin; and most preferably at least five consecutive amino acids from proinsulin.

For reference, a polypeptide sequence of human insulin is reported in the UniProtKB datase (reference P01308) along some of its variants.

Thus, according to some embodiments, the antigen may be in the form of a fusion protein characterised in that it comprises:
an IgG binding moiety consisting of at least two IgG binding domains of streptococcal protein G placed in tandem arrangement;
as a cargo moiety, a polypeptide comprising an antigen-derived sequence such as one selected from the group consisting of insulin, preproinsulin, proinsulin, or an immunologically active fragment thereof.

Regarding diabetes autoantigens specifically, reference is also made to Kratzer et al. (J Immunol 184 (2010): 6855-64) which teaches a P3UmPI fusion protein comprising a proinsulin antigen of murine origin, which can advantageously be substituted by a human insulin, preproinsulin or proinsulin polypeptide sequence.

According to some embodiments, the ratio of antigen vs Nanoparticules (autoantigen/NP) ranges from 1 to about 400; which includes from 1 to about 50; which includes about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50. According to particular embodiments, the ratio (antigen/NP) ranges from 1 to 20, which includes from 3 to 15.

According to some embodiments, the ratio of proinsulin autoantigen vs Nanoparticules (proinsulin/NP) ranges from 1 to about 400; which includes from 1 to about 50; which includes about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50. According to particular embodiments, the ratio (proinsulin/NP) ranges from 1 to 20, which includes from 3 to 15.

AHR Transcription Factor Ligands

Examples of ligands which can bind to an aryl hydrocarbon receptor (AHR) transcription factor, and which are suitable in the context of the invention, include the high affinity AHR ligand 2,3,7,8-tetrachlorodibenzo-p-dioxin (TCDD), tryptamine (TA), and/or 2-(1′H-indole-3′-carbonyl)-thiazole-4-carboxylic acid methyl ester (ITE). Other potential AhR transcription factor ligands are described in Denison and Nagy (Ann. Rev. Pharmacol. Toxicol., 43:309-34, 2003), all of which are incorporated herein in their entirety. Other such molecules include planar, hydrophobic HAHs (such as the polyhalogenated dibenzo-pdioxins, dibenzofurans, and biphenyls) and PAHs (such as 3-methylcholanthrene, benzo(a)pyrene, benzanthracenes, and benzoflavones), and related compounds. (Denison and Nagy, 2003, supra).

Nagy et al., Toxicol. Sci. 65:200-10 (2002), described a high-throughput screen useful for identifying and confirming other ligands. See also Nagy et al. (Biochem. 41:861-68 (2002).

In a non-exhaustive manner, such AHR transcription factor ligands may be selected from indole and metabolites thereof, phytochemicals (e.g. indigorubin or indigo), tryptophane and metabolites thereof, heme-derived compounds (e.g. bilirubin, biliverdin), arachidonic acid and metabolites thereof.

In some embodiments, those ligands useful in the present invention are those that bind competitively with TCDD, TA, and/or ITE.

Most preferably, the ligand which can bind to the aryl hydrocarbon receptor (AHR) transcription factor, is ITE. AhR ligands can also include structural analogs of 2-(1′H-indole-3′-carbonyl)-thiazole-4-carboxylic acid methyl ester (ITE), which are described in WO2016154362.

Accordingly, in some embodiments, the AhR ligands can include compounds having the following formula:

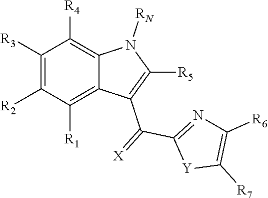

wherein X and Y, independently, can be either O (oxygen) or S (sulfur);

R_{N} can be selected from hydrogen, halo cyano formyl alkyl haloalkyl alkenyl alkynyl, alkanoyl, haloalkanoyl, or a nitrogen protective group;

R₁, R₂, R₃, R₄, and R₅ can be independently selected from hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl, or carbonyloxy;

R₆ and R₇, can be independently selected from hydrogen, halo, hydroxy, thiol, cyano, formyl, alkyl, haloalkyl, alkenyl, alkynyl, amino, nitro, alkoxy, haloalkoxy, or thioalkoxy;

or R₆ and R₇, independently, can be:

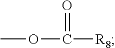

wherein R₈ can be selected from hydrogen, halo, cyano, alkyl, haloalkyl, alkenyl, or alkynyl;

or R₆ and R₇, independently, can be:

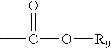

wherein R₉ can be selected from hydrogen, halo, alkyl, haloalkyl, alkenyl, or alkynyl;

or R₆ and R₇, independently, can be:

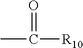

wherein R₁₀ can be selected from hydrogen, halo, hydroxy, thiol, cyano, alkyl, haloalkyl, alkenyl, alkynyl, amino, or nitro;

or R₆ and R₇, independently, can also be:

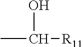

wherein R₁₁ can be selected from hydrogen, halo, alkyl, haloalkyl, alkenyl, or alkynyl.

In some embodiments, the structure of the 2-(1′H-indole-3′-carbonyl)-thiazole-4-carboxylic acid methyl ester analog is represented by one of the following formulas:

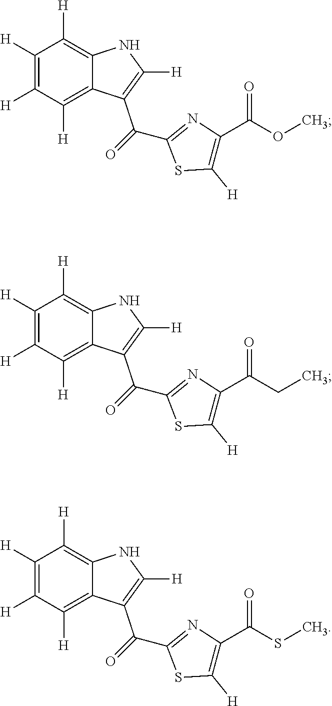 [SMILES: COC(=O)c1csc(C(=O)c2c[nH]c3ccccc23)n1 ; CCC(=O)c1csc(C(=O)c2c[nH]c3ccccc23)n1 ; CSC(=O)c1csc(C(=O)c2c[nH]c3ccccc23)n1]

Other such molecules include polycyclic aromatic hydrocarbons exemplified by 3-methylchoranthrene (3-MC); halogenated aromatic hydrocarbons typified by 2,3,7,8-tetrachlorodibenzo-p-dioxin (TCDD); planar, hydrophobic HAHs (such as the polyhalogenated dibenzo-p-dioxins and dibenzofurans (e.g., 6-methyl-1,3,8-trichlorodibenzofuran or 6-MCDF), 8-methyl-1,3,6-trichlorodibenzofuran (8-MCDF)), and biphenyls) and polycyclic aromatic hydrocarbons (PAHs) (such as 3-methylcholanthrene, benzo(a)pyrene, benzanthracenes, and benzoflavones), and related compounds).

Naturally-occurring AHR ligands can also be used, e.g., tryptophan catabolites such as indole-3-acetaldehyde (IAAlD), indole-3-aldehyde (IAlD), indole-3-acetic acid (IAA), tryptamine (TrA), kynurenine, kynurenic acid, xanthurenic acid, 5-hydroxytryptophan, serotonin; and Cinnabarinic Acid (Lowe et al., PLoS ONE 9(2): e87877; Zelante et al., Immunity 39, 372-385, Aug. 22, 2013; Nguyen et al., Front Immunol. 2014 Oct. 29; 5:551); biliverdin or bilirubin (Quintana and Sherr, Pharmacol Rev 65:1148-1161, October 2013); prostaglandins (PGF3a, PGG2, PGH1, PGB3, PGD3, and PGH2); leukotrienes, (6-trans-LTB 4, 6-trans-12-epi-LTB); dihydroxyeicosatriaenoic acids (4,5(S),6(S)-DiHETE, 5(S),6(R)-DiHETE); hydroxyeicosatrienoic acid (12(R)-HETE) and lipoxin A4 (Quintana and Sherr, Pharmacol Rev 65:1148-1161, October 2013).

In some embodiments, the AHR ligand is a flavone or derivative thereof, e.g., 3,4-dimethoxyflavone, 3′-methoxy-4′-nitroflavone, 4′,5,7-Trihydroxyflavone (apigenin) or 1-Methyl-N-[2-methyl-4-[2-(2-methylphenyl)diazenyl]phenyl-1H-pyrazole-5-carboxamide; resveratrol (trans-3,5,4′-Trihydroxystilbene) or a derivative thereof; epigallocatechin or epigallocatechingallate.

In some embodiments, the AHR ligand is one of the 1,2-dihydro-4-hydroxy-2-oxo-quinoline-3-carboxanilides, their thieno-pyridone analogs, and prodrugs thereof, e.g., having the structure:

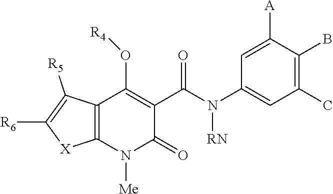

wherein

A, B and C are independently chosen from the group comprising H, Me, Et, iso-Pr, tert-Bu, OMe, OEt, O-iso-Pr, SMe, S(O)Me, S(O)2 e, CF3, OCF3, F, Cl, Br, I, and CN, or A and B represents OCH₂O and C is H;

RN is chosen from the group comprising H, C(O)H, C(O)Me, C(O)Et, C(O)Pr, C(O)CH(Me)₂, C(O)C(Me)₃, C(O)Ph, C(O)CH₂Ph, CO₂H, CO₂Me, CO₂Et, CO₂CH₂Ph,

C(O)NHMe, C(O)NMe₂, C(O)NHEt, C(O)NEt₂, C(O)NHPh, C(O)NHCH₂Ph, the acyl residues of C₅-C₂₀ carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising Me, Et, OMe, OEt, SMe, S(O)Me, S(O)₂Me, S(O)₂NMe₂, CF₃, OCF₃, F, Cl, OH, CO₂H, CO₂Me, CO₂Et, C(O)NH₂, C(O)NMe₂, NH₂, NH₃, NMe₂, NMe₃+, NHC(O)Me, NC(═NH)NH₂, OS(O)₂OH, S(O)₂OH, OP(O)(OH)₂, and P(O)(OH)₂;

R₄ is RN, or when RN is H, then R₄ is chosen from the group comprising H, P(O) (OH)₂, P(O) (OMe)₂, P(O) (OEt)₂, P(O) (OPh)₂, P(O) (OCH₂Ph)₂, S(O)₂OH, S(O)₂NH₂, S(O)₂NMe₂, C(O)H, C(O)Me, C(O)Et, C(O)Pr, C(O)CH(Me)₂, C(O)C(Me)₃, C(O)Ph, C(O)CH₂Ph, CO₂H, CO₂Me, CO₂Et, CO₂CH₂Ph, C(O)NHMe, C(O)NMe₂, C(O)NHEt, C(O)NEt₂, C(O)NHPh, C(O)NHCH₂Ph, the acyl residues of C₅-C₂₀ carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising Me, Et, OMe, OEt, SMe, S(O)Me, S(O)₂Me, S(O)₂NMe₂, CF₃, OCF₃, F, Cl, OH, CO₂H, CO₂Me, CO₂Et, C(O)NH₂, C(O) Me₂, NH₂, NH₃+, Me₂, NMe₃+, NHC(O)Me, NC(═NH)NH₂, OS(O)₂₀H, S(O)₂₀H, OP(O) (OH)₂, and P(O)(OH)₂;

R₅ and R₆ are independently chosen from the group comprising H, Me, Et, iso-Pr, tert-Bu, OMe, OEt, O-iso-Pr, SMe, S(O)Me, S(O)₂Me, CF₃, OCF₃, F, Cl, Br, I, and CN, or R₅ and R₆ represents OCH₂O; and X is —CH═CH—, or S, or pharmaceutically acceptable salts of the compounds thereof.

In some embodiments, the AHR ligand is laquinimod (a 5-Cl, N-Et carboxanilide derivative) or a salt thereof (see, e.g., US20140128430).

In some embodiments, the AHR ligand is characterized by the following general formula:

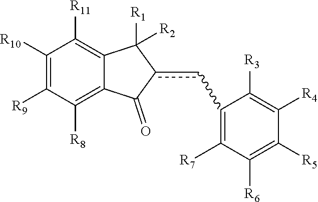

wherein

(i) R₁ and R₂ independently of each other are hydrogen or a C₁ to C₁₂ alkyl,

(ii) R₃ to R₁₁ independently from each other are hydrogen, a C₁ to C₁₂ alkyl, hydroxyl or a C₁ to C₁₂ alkoxy, and

(iii) the broken line represents either a double bond or two hydrogens.

In some embodiments, the AhR ligand has one of the following formulae:

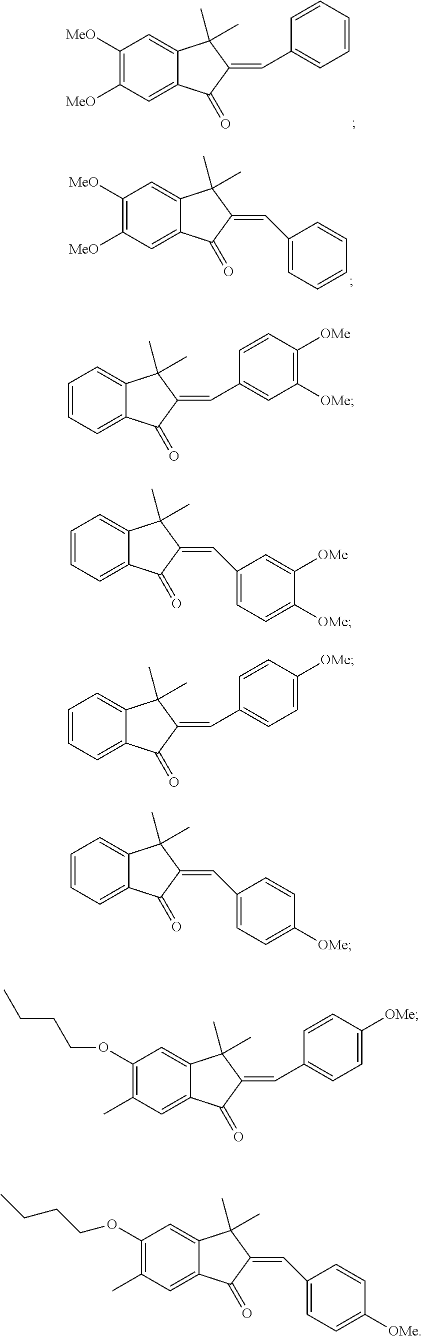

In some embodiments, the AhR ligand has a general formula of:

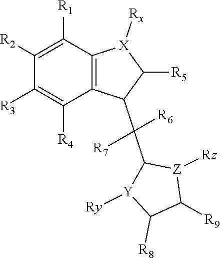

wherein:

R₁, R₂, R₃ and R₄ can be independently selected from the group consisting of hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl and carbonyloxy.

R₅ can be selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.

R₆ and R₇ together can be ═O.

Alternatively, R₆ can be selected from the group consisting of hydrogen, halo, cyano, formyl, alkyl, haloalkyl, alkenyl, alkynyl, alkanoyl and haloalkanoyl, and R₇ is independently selected from the group consisting of hydrogen, halo, hydroxy, thiol, cyano, formyl, alkyl, haloalkyl, alkenyl, alkynyl, amino, nitro, alkoxy, haloalkoxy, thioalkoxy, alkanoyl, haloalkanoyl and carbonyloxy.

Alternatively, R₇ can be selected from the group consisting of hydrogen, halo, cyano, formyl, alkyl, haloalkyl, alkenyl, alkynyl, alkanoyl and haloalkanoyl, and R₆ is independently selected from the group consisting of hydrogen, halo, hydroxy, thiol, cyano, formyl, alkyl, haloalkyl, alkenyl, alkynyl, amino, nitro, alkoxy, haloalkoxy, thioalkoxy, alkanoyl, haloalkanol and carbonyloxy.

R₈ and R₉, independently, can be

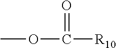

and R₁₀ is selected from the group consisting of hydrogen, halo, cyano, alkyl, haloalkyl, alkenyl and alkynyl.

Alternatively, R₈ and R₉, independently, can be

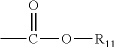

and R₁₁ is selected from the group consisting of hydrogen, halo, alkyl, haloalkyl, alkenyl and alkynyl.

Alternatively, R₈ and R₉, independently, can be

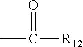

and R₁₂ is selected from the group consisting of hydrogen, halo, hydroxy, thiol, cyano, alkyl, haloalkyl, alkenyl, alkynyl, amino and nitro.

Alternatively, R₈ and R₉, independently, can be

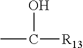

and R₁₃ is selected from the group consisting of hydrogen, halo, alkyl, haloalkyl, alkenyl and alkynyl.

Alternatively, R₈ and R₉, independently, can be selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.

X can be oxygen or sulfur, and Rx is nothing. Alternatively, X can be nitrogen, and Rx is selected from the group consisting of hydrogen, halo, formyl, alkyl, haloalkyl, alkenyl, alkynyl, alkanoyl, haloalkanoyl and a nitrogen protective group. Alternatively, X can be carbon, and Rx is selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.

Y can be oxygen or sulfur, and Ry is nothing. Alternatively, Y can be nitrogen, and Ry is selected from the group consisting of hydrogen, halo, formyl, alkyl, haloalkyl, alkenyl, alkynyl, alkanoyl, haloalkanoyl and a nitrogen protective group. Alternatively, Y can be carbon, and Ry is selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.

Z can be oxygen or sulfur, and Rz is nothing. Alternatively, Z is nitrogen, and Rz is selected from the group consisting of hydrogen, halo, formyl, alkyl, haloalkyl, alkenyl, alkynyl, alkanoyl, haloalkanoyl and a nitrogen protective group.

Alternatively, Z can be carbon, and Rz is selected from hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.

Other AHR ligands include stilbene derivatives and flavone derivatives of formula I and formula II, respectively:

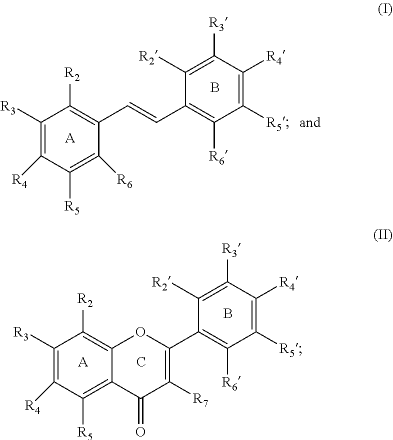

wherein R₂, R₃, R₄, R₅, R₆, R₇ and R₂′ R₃′, R₄′, R₅′, R₆′ are identical or different (including all symmetrical derivatives) and represent H, OH, R (where R represents substituted or unsubstituted, saturated or unsaturated, linear or branched aliphatic groups containing one to thirty carbon atoms), Ac (where Ac represents substituted or unsubstituted, saturated or unsaturated, cyclic compounds, including alicyclic and heterocyclic, preferably containing three to eight atoms), Ar (where Ar represents substituted or unsubstituted, aromatic or heteroaromatic groups preferably containing five or six atoms), Cr (where Cr represents substituted or unsubstituted fused Ac and/or Ar groups, including Spiro compounds and norbornane systems, preferably containing two to five fused rings), OR, X (where X represents an halogen atom), CX₃, CHX₂, CH₂X, glucoside, galactoside, mannoside derivates, sulfate and glucuronide conjugates. Optical and geometrical isomeric derivatives of stilbene and flavone compounds are included.

B_{reg} Cells-Enriched Composition

Also provided herein are pharmaceutical compositions and formulations comprising stimulated B_{regs} and a pharmaceutically acceptable carrier.

Pharmaceutical compositions and formulations as described herein can be prepared by mixing the active ingredient (the B_{reg} cells-enriched composition) having the desired degree of purity with one or more optional pharmaceutically acceptable carriers (Remington's Pharmaceutical Sciences 22nd edition, 2012), in the form of lyophilized formulations or aqueous solutions.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968.

### Examples

### A. Materials & Methods

Materials: Reagents for particle synthesis were from Sigma-Aldrich (Saint Louis, Mo., USA); Phosphonate-poly(ethylene glycol) PO-PEG-COOH (SP-1P-10-002, MW 2500 g·mol⁻¹) was purchased from Specific Polymers (Specific polymers, Castries, France). The 2-(1H-Indol-3-ylcarbonyl)-4-thiazolecarboxylic acid methyl ester (ITE) was purchased from Tocris bioscience (Bristol, United Kingdom). 1-(3-Dimethylaminopropyl)-3-ethylcarbodiimide hydrochloride (EDC) was purchased from Alfa Aesar (Karlsruhe, Germany).

Fusion protein expression and purification: The fusion protein P₃UmPI is expressed in BL21DE3 pET16b bacteria. Bacteria are pre-cultured at 37° C. in 20 mL LB broth purchased from Sigma-Aldrich (Saint Louis, Mo., USA). 5 mL of the preculture is cultured 4 hours in 500 mL LB broth, ampicillin (50 μg/mL). Protein expression is induced during 4 hours by Isopropyl-13-D-thiogalactoside from Sigma-Aldrich (Saint Louis, Mo., USA). Bacteria are then pelleted by centrifugation (10 min, 5000 g, 4° C.). The pellet is lysed 30 min on ice in a lysis buffer (Tris 50 mM, NaCl 50 mM, TCEP 1 mM, EDTA 0.5 mM, glycerol 5%, pH8), lysozyme 0.2 mg/mL and DNase I 0.1 mg/mL. The mix is sonicated 10 times with 1 min off/on pulse. Triton 1% is added for 15 min and after centrifugation (20,000×g, 1H, 4° C.) the supernatant is passed over a rabbit IgG-Sepharose column. Protein is eluted with a CHAPS1%/CAPS 20 mM buffer. Then the protein is dialyzed overnight (MWCO 8000 kDa) in PBS, glycerol 10%. Finally, the protein is passed through columns for removal of detergent (Pierce™ Detergent Removal Spin Column; Thermofisher Scientific, Waltham, Mass.) and endotoxin (Endotoxin Removal Spin Column; Thermofisher). Concentration is then measured with fluorescent assay on Qubit (Thermo fisher).

USPIO-PO-PEG-COOH NP synthesis and surface functionalization: 9 nm non-coated NPs were synthesized by the reaction of Iron (III) acetyl acetonate (1.1 mmol) with 10 ml of benzylalcohol at 250° C. during 30 min under microwave irradiation on a Monowave 300 from Anton Paar (Anton-Paar, GmbH, Graz, Austria). The resulting suspension was separated using a neodymium magnet, and the precipitate was washed sequentially with dichloromethane followed by sodium hydroxide solution 1 mol·l⁻¹ and finally ethanol 90% (three times for each wash buffer). The solid was re-dispersed in pH=2 water using an HCl solution at 10⁻¹ mol·l⁻¹ and washed three times by ultracentrifugation (Amicon 30 kDa, Merck Millipore). To coat the NPs with PO-PEG-COOH, both compounds were mixed at pH=2 with an equivalent mass (PO-PEG-COOH/NP) ratio of 10. Then the excess of PO-PEG-COOH coating molecules is removed using ultrafiltration (Amicon 30 kDa, Merck Millipore) and USPIO-PO-PEG-COOH NPs were dispersed in water (Invitrogen™ UltraPure™ DNase/RNase-Free Distilled Water pH=7), Sodium chloride solution 0.9% in water (BioXtra), or NaCl 0.9% (BioXtra)/Glucose (Sigma) 5% and the pH was adjusted at 7.4 using NaOH (10⁻¹ mol·l⁻¹) solutions.

Coupling onto USPIO-PO-PEG-COOH NPs: To load ITE on NPs, ITE in DMSO was added to USPIO-PO-PEG-COOH NPs in water at room temperature with a ratio RITE/NP=600. After 2 hours under mixing, NPs are washed by ultracentrifugation three times for 15 min (Amicon 30 kDa, Merck Millipore). The coupling of P₃UmPI (37.8 kDa) onto USPIO-PO-PEG-COOH NPs was performed in coupling buffer (PF127 3 g/L, H₃PO₄ 0.5 μmol·L⁻¹, pH=6) in a two-step procedure (activation and conjugation) at 37° C. First, the carboxylic acid functions at the outer surface of the NPs were activated using 1-ethyl-3-(3-dimethylaminopropyl) carbodiimide (EDC, n_{EDC}=5n_{COOH}) at pH=6 during 10 min. The second step was the linkage of the amine function of the protein with the activated carboxylic acid functions on the NPs. The protein is added at pH=6 to the ferrofluid during 1 h at 37° C. The procedure was carried out with the ratio R=n_{P3UmPI}/n_{NP}=8. The NPs are washed by ultracentrifugation three times for 15 min (Amicon 100 kDa, Merck Millipore). The NPs were re-dispersed in water at physiological pH for various physicochemical characterizations.

Physico-chemical characterization: The hydrodynamic size and zeta potential of the NPs ([Fe]=0.25 mM) were investigated by dynamic laser light scattering (DLS), using a Nano-ZS (Red Badge) ZEN 3600 device (Malvern Instruments, Malvern, UK). The stability in physiological medium (NaCl 0.9% and NaCl 0.9%/Glucose 5%, [Fe]=0.25 mM) was studied by measuring over time the evolution of the hydrodynamic size.

UV-Vis spectra were recorded on a Varian Cary 50 Scan UV-vis spectrophotometer. TEM images were obtained using a FEI Tecnai 12 (Philips), and samples were prepared by depositing a drop of NP suspension on carbon-coated copper grids placed on a filter paper. The median diameter is deduced from TEM data measurements, simulating the diameter distribution with a log-normal function, according to the methodology described in de Montferrand et al. (“Size-Dependent Nonlinear Weak-Field Magnetic Behavior of Maghemite Nanoparticles”; Small; 2012; 8(12), 1945-56). The grafting of the PO-PEG-COOH to the surface of the NPs, the ITE loading and the coupling of protein P3UmPI was studied by Fourier transform infra-red (FTIR) analysis.

The FTIR spectra were recorded as thin films on KBr pellets on a Thermo Scientific Nicolet 380 FTIR. Quantification of PO-PEG-COOH coating and grafting per particle was evaluated by thermogravimetric analysis (TGA) using a LabsSys evo TG-DTA-DSC 16000 device from Setaram Instrumentation.

The average number of ITE per NP was evaluated using infrared and UV-Visible spectroscopies. For the infrared spectroscopy method, infrared spectra in KBr pellets of various proportions of ITE mixed with a constant amount of USPIO-PO-PEG-COOH NPs were recorded. Then, the normalized 1735 cm⁻¹ band was used for the establishment of a calibration curve and the average number ITE per nanoparticle was deduced from this curve. For the UV-Visible spectroscopy method: ITE saponification was performed by adding 2 mL NaOH 1 mol·L⁻¹ to ITE or ITE-loaded NP for 2 h (2004, of a NP [Fe]=3.5 μM). In the latter case, the NPs were isolated from supernatant using magnetic decantation. The resulting carboxylate ion (carboxylate ITE) was water soluble and characterized by two UV bands at 279 and 388 nm. A calibration curve was established after basic hydrolysis of ITE alone and the average number ITE per nanoparticle was deduced from this curve.

The ITE saponification was characterized with NMR experiments. 1H NMR spectra (400 MHz, 258C), were recorded in D₂O on a Bruker Avance 400 spectrometer and chemical shifts are reported in parts per million (ppm) on the δ scale.

The coupling efficiency of the fusion protein P₃UmPI conjugation was investigated qualitatively using the o-phthalaldehyde (OPA) method. 50 μL of the sample was diluted in 50 μL of NaOH 2 mol·L⁻¹ and left overnight at 60° C. NPs were separated from supernatant using magnetic decantation. 900 μL of OPA reagent was added to the supernatant and fluorescence measurement at 450 nm was recorded on a SpectroFluorimeter Spex FluoroMax (HORIBA Jobin-Yvon, France with a Hamamatsu 98 photomultiplier). The average number of protein per nanoparticle was deduced from a calibration curve.

Using UV-visible spectroscopy and the o-phthalaldehyde method, respectively, complete NPs were determined to contain 348±70 molecules ITE and 4.3 molecules P3UmPI per particle.

When the nanoparticules are iron, magnetic, nanoparticles, the iron concentration can be determined by a colorimetric assay as described in Richard et al. (ACS Chem Biol, 2016, 11 (10), 2812-2819). A Vibrating Sample Magnetometer (VSM Quantum Design, Versalab) was used for magnetic characterization. VSM measures the magnetization by cycling the applied field from −30 to +30 kOe with a step rate of 100 Oe·s⁻¹. Measurements were performed on USPIO solutions at [Fe]=12.5 mM (corresponding to [NP]=0.8 μM and [Fe₂O₃]=1 g·l⁻′) at 300 K. The ZFC curve was obtained by first cooling the system in zero field from 270 K to 50 K. Next, an external magnetic field of 100 Oe was applied, and subsequently the magnetization was recorded with a gradual increase in temperature. The FC curve was measured by decreasing the temperature in the same applied field.

In vitro B cell treatment: B cells were magnetically isolated using MojoSort™ Mouse Pan B cell Isolation Kit (Biolegend). The resulting B cells (6×10⁵ cells per well) from 11-weeks-old female NOD or C57BL/6J mice were incubated for 72 h in complete IMDM with 90 μmol of Fe equivalent of NP-PEG-ITE-P3UmPI or vehicle. For cytokine detection, cells were incubated with LPS and in the presence of a protein transport inhibitor (eBioscience) for the last 5 h.

Transfer Experiments

NOD Rag^{-/-} mice (aged 8 to 10 weeks) were i.v. injected with 10×10⁶ splenic T cells sorted from diabetic NOD mice, using a biotinylated mouse TCR β chain antibody (Biolegend, H57-597) and anti-biotin microbeads (Miltenyi). As indicated, 10×10⁶ sorted splenic B cells treated ex vivo for 72 h with complete NPs or sorted from NOD mice previously treated with NP-PEG or NP-PEG-ITE-P3UmPI in vivo (3 injections during 10 days) were co-transferred (10×10⁶). T1D incidence was monitored starting 2 weeks post-transfer.

Flow Cytometry

Single cell suspensions were stained for 30 min at 4° C. after FcγRII/III blocking with anti-CD16/CD32 monoclonal antibody (mAb).

Staining buffer was PBS containing 2% FCS, 0.5% EDTA and 0.1% sodium azide. Surface staining was performed with mAbs recognizing: CD45 (eBioscience, 30-F11), CD11b (eBioscience, M1/70), F4/80 (eBioscience, BM8), CD11c (eBioscience, N418), TCRβ (eBioscience, H57-597), CD19 (eBioscience, 1D3), (Biolegend, C068C2), CD5 (Miltenyi, 53-7.3), CD1d (Biolegend, K253), CD21 (Biolegend, 7E9), B220 (Biolegend, RA3-6B2), CD138 (Biolegend, 281-2), CD86 (Biolegend, GL-1), CD8a (eBioscience, 53-6.7), CD4 (Biolegend, RM4-5), CD23 (Biolegend, B3B4), CD44 (eBioscience, IM7), CD62L (Biolegend, MEL-14). For measurement of active TGFβ, cells were surface stained with anti-LAP mAb (eBioscience, TW7-16B4).

To measure cytokine expression, cell suspensions were incubated 5 h at 37° C. with the relevant stimulus (LPS for B cells, PMA ionomycin for T cells), in the presence of a protein transport inhibitor (eBioscience), surface stained, fixed and then intracellularly stained using the intracellular staining kit (Biolegend). Further details including antibodies used for flow cytometry are given in the SM.

Statistical analysis: Diabetes incidence was plotted according to the Kaplan-Meier method. Incidences between different groups were compared with the log-rank test. Reported values are mean+/−standard deviation. Comparisons between different groups were performed using the two-way ANOVA test. P values <0.05 were considered statistically significant. All data were analyzed using GraphPad Prism v6 software.

### B. Results

B.1 Effect of Short-Term NP Treatment on B Cells Ex Vivo.

Having observed strong effects in vivo associated to the administration of complete NPs on splenic B cells and myeloid cells, we wondered whether induction of regulatory B was a direct effect or required the presence of other populations. To address this, we sorted splenic B cells from prediabetic NOD and C57BL/6 mice, and incubated them in vitro for 3 days with NPs and analyzed phenotype and effector functions.

While TGF-β and IDO induction was stronger for NOD cells, surprisingly C57BL/6 B cells responded with higher numbers of IL-10 producers to this treatment. Complete NPs induced IL-10 (FIG. 1A) and TGF-β, the former equally well in both strains but the latter much stronger in NOD B cells; IL-4 and IDO also seemed to be increased exclusively in NOD B cells though at levels below statistical significance.

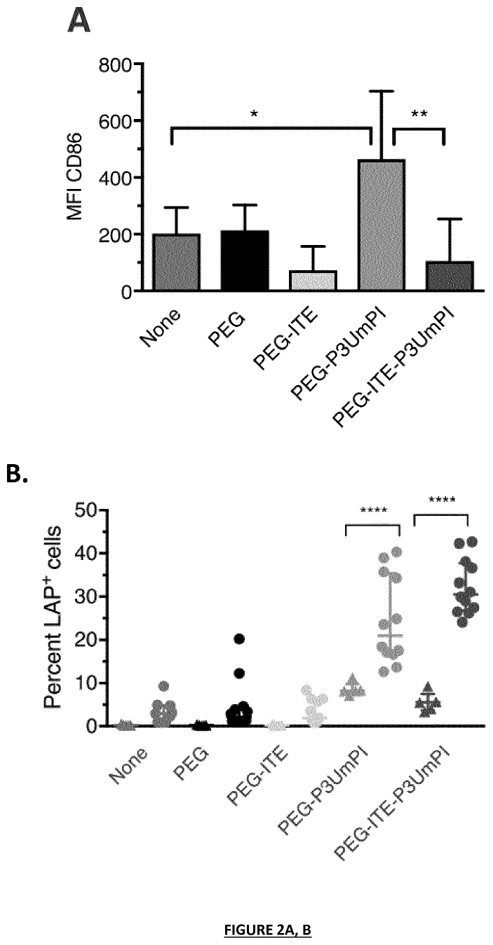

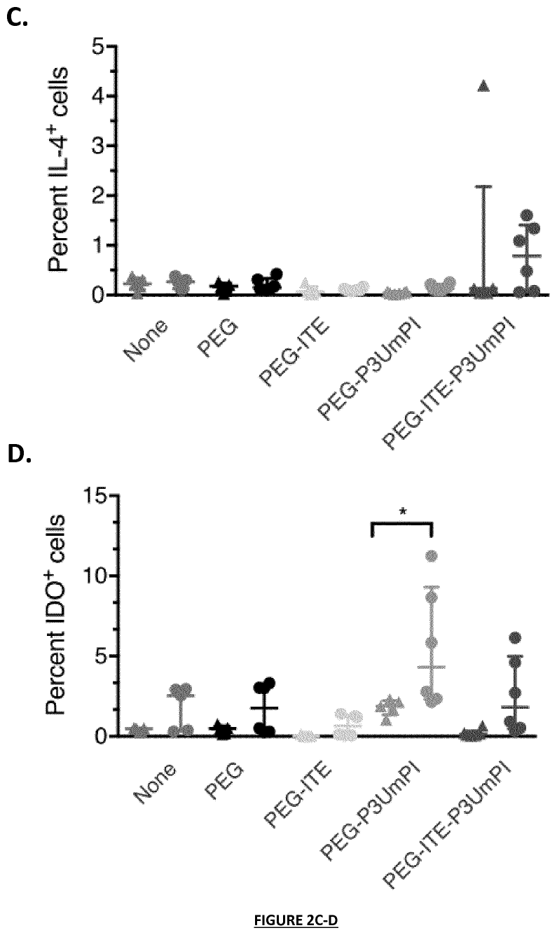

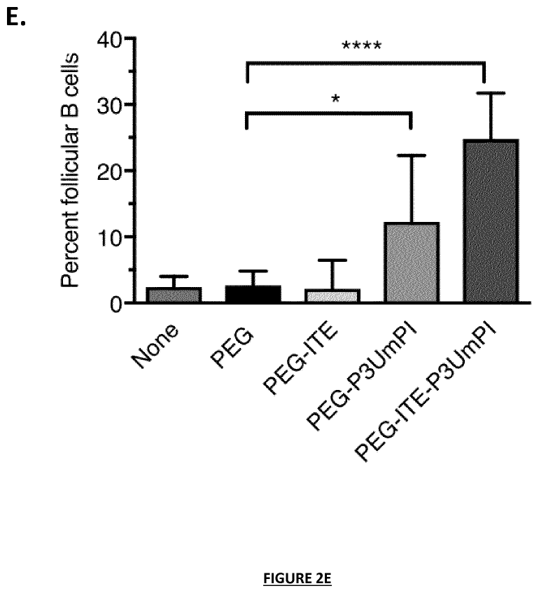

Globally the most pronounced effect of ex vivo treatment was induction of TGF-β in NOD B cells by PEG-P3UmPI and ITE-P3UmPI NPs (the latter being also referred herein as “complete nanoparticles (NPs)”), with up to 30% of cells producing the regulatory cytokine (FIG. 2B). Both NP types also induced substantial and up to 12-fold expansion of follicular B cells (FIG. 2E).

NP-loaded ITE and P3UmPI had opposite effects on B cell activation, with an increase by P3UmPI that was abolished by a dominant decrease mediated by ITE (FIG. 2A). Both NPs carrying P3UmPI alone and complete NPs were able to induce regulatory cytokines. P3UmPI-NPs induced IL-10, TGF-β and IDO (FIG. 1A, 2B, 2D).

The strongest effect of complete NPs clearly targeted B lymphocytes. This effect was evident both upon a 10-day treatment of pre-diabetic mice and upon a 3-day in vitro treatment of sorted B cells and included B cell activation, expansion of follicular B cells and production of regulatory B cells producing IL-10, TGF-β, IL-4 and IDO.

Interestingly, the results obtained in vitro suggested that ITE may not be required for the latter effect (FIG. 2A).

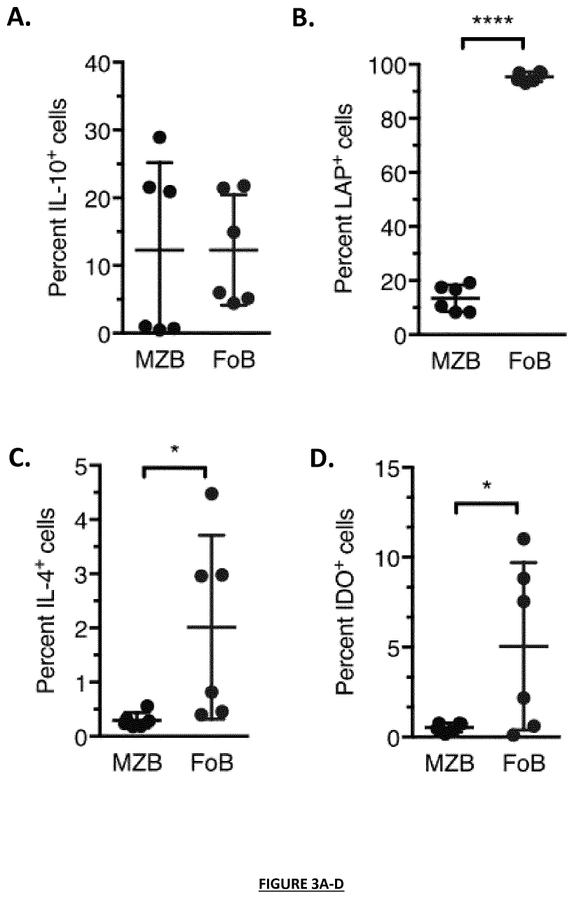

While IL-10 was produced by equal proportions of marginal zone and follicular B cells, the latter were almost entirely responsible for producing TGF-β, IL-4 and IDO, with up to 95% of follicular B cells expressing TGF-β (FIG. 3).

Overall, this data shows that the described nanoparticles are suitable for stimulating the production of Interleukin-10 in vitro or ex vivo (FIG. 1A), for stimulating the production of TGF-β in vitro or ex vivo (FIG. 2B) and for producing regulatory B cells ex vivo (FIGS. 1A & 2 as a whole).

B2. In Vivo Effect of Regulatory B Cells Produced Ex Vivo

We wondered whether regulatory B cells produced ex vivo were able to delay or prevent T1D. To address this, we adoptively transferred splenic T cells obtained from recently diabetic NOD mice, together with sorted B cells from the spleen of NP-treated prediabetic NOD mice, or with B cells incubated with complete NPs ex vivo, to immunodeficient NOD Rag^{-/-} mice and monitored diabetes occurrence. Co-transfer of B cells from mice treated with PEG NPs did not delay diabetes appearance due to diabetogenic T cells, with all mice being diabetic by 45 days after transfer. However, B cells treated in vivo or ex vivo with complete NPs delayed disease, such that 100% diabetes was reached only on day 80 or 75, respectively (FIG. 1B).

B cells treated ex vivo conferred the strongest protection, with a mean delay of 61 days vs. 49 for B cells treated with complete NPs in vivo, 33 for T cells alone and 37 for PEG-NP-treated B cells. We concluded that short-term ex vivo treatment of splenic B cells with NPs containing P3UmPI alone or together with ITE induces expansion of follicular B cells producing regulatory cytokines, especially TGF-β, capable of inhibiting disease transfer by diabetogenic T cells.

Overall, this data (FIG. 1B) shows that the ex vivo produced regulatory B cells provide a higher capacity to delay the appearance of the disease.

B.3 Splenic and PLN Immune Populations in Mice Cured Upon NP Treatment

Our observations so far suggested a major role for regulatory B cells in the short-term effects of NP treatment. Wondering whether this role extended to the long-term effects of NPs, we compared splenic and PLN immune cell populations in the two mice in stable remission for >300 days after treatment, to the equivalent populations in non-autoimmune and in pre-diabetic and diabetic NOD mice. While the spleens of untreated NOD mice contained greater numbers of CD45⁺, TCR-β⁺, CD4⁺ and CD8⁺ T cells than control C57BL/6 mice, the size of these populations was lower and identical to control mice in treated animals. Remarkably, the reduced cellularity extended to splenic B cells and DCs/macrophages, two populations found increased upon short-term treatment.

The most striking result in the two “cured” mice was the string emergence of Foxp3+ T cells

Overall, this data shows that the ex vivo produced regulatory B cells also provide a long-term effect, in the context of a treatment of T1D.

1. An in vitro or ex vivo method for increasing the number of B regulatory (B_{reg}) cells in a population of B cells, the method comprising:
(i) providing a population of isolated B cells;
(ii) bringing into contact the population of isolated B cells with an efficient amount of a biocompatible nanoparticle comprising at least one antigen, thereby increasing the number of B_{reg} cells in the population, thereby providing a B_{reg} cells-enriched composition;
(iii) optionally recovering B_{reg} cells from the B_{reg} cells-enriched composition.

2. An in vitro or ex vivo method for producing Interleukin-10 (IL-10) or TGF-β, the method comprising:
(i) providing a population of isolated B cells;
(ii) bringing into contact the population of isolated B cells with an efficient amount of a biocompatible nanoparticle comprising at least one antigen, thereby producing Interleukin-10 or TGF-β;
(iii) optionally recovering the Interleukin-10 or TGF-β, from step (ii).

3. The method according to claim 1, wherein the at least one antigen is an autoantigen.

4. The method according to claim 1 wherein the at least one antigen is an autoantigen selected from: Carboxypeptidase H, Chromogranin A, Glutamate decarboxylase, Imogen-38, Insulin, Insulinoma antigen-2 (IA-2) and 2β, Islet-specific glucose-6-phosphatase catalytic subunit related protein (IGRP), Proinsulin, Preproinsulin, Glutamate Decarboxylase (GAD), Zinc-Transporter 8 (ZnT8), Chromogranin A, α-enolase, Aquaporin-4, β-arrestin, Myelin basic protein (MBP), Myelin Oligodendrocytic Glycoprotein (MOG), Myelin Proteolipid Protein (PLP), Myelin Associated Glycoprotein (MAG), Myeline-associated Oligodendrocyte Basic Protein (MOBP), 2′,3′-Cyclic-nucleotide 3′-phosphodiesterase (CNPase), S100-β10 (S100-β), nAChR, MuSK, LRP4, Citrullinated antigen, Carbamylated antigen, Collagen such as Collagen type I, Collagen type II, Collagen type III, Collagen type IV, Heat shock proteins such as 6(-kDa heat-shock protein, Human cartilage glycoprotein 39, Double-stranded DNA, La antigen, Nucleosomal histones and ribonucleoproteins (snRNP), Phospholipid-β-2 glycoprotein I complex, Poly(ADP-ribose) polymerase, Sm antigens of U-1 small ribonucleoprotein complex11, Transaldolase, Fc-part of immunoglobulins, Aggrecan G1, Aquaporin 4 (AQP-4), NMDA-receptor, AMPA-receptor, GABA receptor, Gly-receptor, Dipeptidyl aminopeptidase-like Protein 6 (DPPX), GluR5, VGKC-complex, HU, Jo, Ri, Ma1, Ma2, Zic4, CRMP5, Amphiphysin; or a immunologically active fragment thereof.

5. The method according to claim 1, wherein the at least one antigen is a diabetes autoantigen selected from: insulin, preproinsulin, proinsulin, or an immunologically active fragment thereof.

6. The method according to claim 1, wherein the biocompatible nanoparticle is a tolerogenic biocompatible nanoparticle.

7. The method according to claim 1, wherein the biocompatible nanoparticle is a tolerogenic biocompatible nanoparticle comprising at least a ligand which can bind to an aryl hydrocarbon receptor (AHR) transcription factor.

8. The method according to claim 1, wherein the biocompatible nanoparticle is a tolerogenic biocompatible nanoparticle comprising at least one ligand which can bind to an aryl hydrocarbon receptor (AHR) transcription factor that is 2-(1′H-indole-3′-carbonyl)-thiazole-4-carboxylic acid methyl ester (ITE).

9. The method according to claim 1 wherein the said nanoparticle has an average size of less than about 60 nm; and preferably less than 20 nm.

10. A composition containing a biocompatible nanoparticle comprising at least one antigen; in combination with a population of isolated B cells.

11. The composition according to claim 10, wherein the biocompatible nanoparticle comprising at least one antigen is present in an injectable solution.

12. A kit comprising:
a biocompatible nanoparticle comprising at least one antigen; and
a population of isolated B cells.

13. A B_{reg} cells-enriched composition obtained by the method of claim 1, or recovered B_{reg} cells thereof.

14. The A therapeutic method comprising a step of administering, to a subject in need thereof, a B_{reg} cells-enriched composition of claim 13, or recovered B_{reg} cells thereof.

15. A method for producing B regulatory (B_{reg}) cells in vivo or for producing Interleukin-10 (IL-10) or TGF-β in vivo, comprising a step of administering a biocompatible nanoparticle comprising at least one antigen.

16. The method according to claim 2, wherein the at least one antigen is an autoantigen.

17. The method according to claim 2 wherein the at least one antigen is an autoantigen selected from: Carboxypeptidase H, Chromogranin A, Glutamate decarboxylase, Imogen-38, Insulin, Insulinoma antigen-2 (IA-2) and 2β, Islet-specific glucose-6-phosphatase catalytic subunit related protein (IGRP), Proinsulin, Preproinsulin, Glutamate Decarboxylase (GAD), Zinc-Transporter 8 (ZnT8), Chromogranin A, α-enolase, Aquaporin-4, β-arrestin, Myelin basic protein (MBP), Myelin Oligodendrocytic Glycoprotein (MOG), Myelin Proteolipid Protein (PLP), Myelin Associated Glycoprotein (MAG), Myeline-associated Oligodendrocyte Basic Protein (MOBP), 2′,3′-Cyclic-nucleotide 3′-phosphodiesterase (CNPase), S100-β10 (S100-β), nAChR, MuSK, LRP4, Citrullinated antigen, Carbamylated antigen, Collagen such as Collagen type I, Collagen type II, Collagen type III, Collagen type IV, Heat shock proteins such as 6(-kDa heat-shock protein, Human cartilage glycoprotein 39, Double-stranded DNA, La antigen, Nucleosomal histones and ribonucleoproteins (snRNP), Phospholipid-β-2 glycoprotein I complex, Poly(ADP-ribose) polymerase, Sm antigens of U-1 small ribonucleoprotein complex11, Transaldolase, Fc-part of immunoglobulins, Aggrecan G1, Aquaporin 4 (AQP-4), NMDA-receptor, AMPA-receptor, GABA receptor, Gly-receptor, Dipeptidyl aminopeptidase-like Protein 6 (DPPX), GluR5, VGKC-complex, HU, Jo, Ri, Ma1, Ma2, Zic4, CRMP5, Amphiphysin; or a immunologically active fragment thereof

18. The method according to claim 2, wherein the at least one antigen is a diabetes autoantigen selected from: insulin, preproinsulin, proinsulin, or an immunologically active fragment thereof.

19. The method according to claim 2, wherein the biocompatible nanoparticle is a tolerogenic biocompatible nanoparticle.

20. The method according to claim 2, wherein the biocompatible nanoparticle is a tolerogenic biocompatible nanoparticle comprising at least a ligand which can bind to an aryl hydrocarbon receptor (AHR) transcription factor.

21. The method according to claim 2 wherein the said nanoparticle has an average size of less than about 60 nm; and preferably less than 20 nm.

22. A B_{reg} cells-enriched composition obtained by the method of claim 3, or recovered B_{reg} cells thereof.

23. A therapeutic method comprising a step of administering, to a subject in need thereof, a B_{reg} cells-enriched composition of claim 22, or recovered B_{reg} cells thereof.
